(19) United States
(12) Plant Patent Application Publication
(10) Pub. No.: US 2007/0044185 P1
(43) Pub. Date: Feb. 22, 2007

(54) Grape plant named 'Improved Chancellor'

(76) Inventors: Robert M. Skirvin, Champaign, IL (US); Margaret A. Norton, Urbana, IL (US); Stephen K. Farrand, Seymour, IL (US); Richard M.S. Mulwa, Msambweni (KE)

Correspondence Address: GREENLEE WINNER AND SULLIVAN P C, 4875 PEARL EAST CIRCLE, SUITE 200, BOULDER, CO, 80301, US

(21) Appl. No.: 11/206,645

(22) Filed: Aug. 17, 2005

(51) Int. Cl. A01H 5/00

(52) U.S. Cl. PLT/205

(57) ABSTRACT

A new and distinct transgenic grape plant Vitis vinifera called “Improved Chancellor” which is characterized by greater tolerance to 2,4-diphenoxyacetic acid than is the parent Chancellor grape plant.

Genus: Vitis (complex interspecific hybrid lineage)

### BACKGROUND AND SUMMARY OF THE INVENTION

The new grape plant named ‘Improved Chancellor’ is of Vitis parentage, with the breeding chart tracing the lineage as shown

[image]

The new grapevine resulted from stably introducing a plant expressible tfdA gene from Ralstonia eutrophus into Chancellor grapes. The tdfA gene confers resistance in plants to the phenoxy herbicides, especially 2,4 D (2,4-diphenoxyacetic acid). For a general discussion, see US Patent Publication No. 2003-0154507. This resistance improves the performance of grapevines, which are by nature exquisitely sensitive to the phenoxy herbicides, with deleterious effects observed with drift of herbicide from fields in the area, especially grain fields sprayed to control broadleaf weeds. Damage from drift negatively affects growth and/or yield in affected vineyards.

Like the parent non-genetically modified (parent) ‘Chancellor’ grape plant, fruit ripening is expected in the same period as the parent in Illinois, about August 30 to September 15 (Skirvin, et al., 2005). Upon ripening, the fruit is expected to contain a sugar level of about 20-21 degrees brix. Data related to fruiting and fruit quality are not currently available.

### DESCRIPTION OF THE INVENTION

The transgenic 2,4-D resistant ‘Chancellor’ grape was produced via genetic engineering. Embryogenic callus was initiated from ovary explants dissected from flower buds 10-14 days before anthesis on Nistch and Nistch (1969) medium containing 9 μM 2,4-D, 17 μM IASP, and 1 μM BAP in darkness. Embryogenic callus was proliferated and maintained in NN medium containing 2 μM 2,4-D, 0.2 μM TDZ and 4 μM IASP (Long Term Maintenance Medium, LTMM). Embryogenic callus cultivated for 5 weeks in LTMM was infected with Agrobacterium tumefasiens strain LBA4404:pAL4404 carrying a pBIN19 plant transformation plasmid vector containing the tfdA gene construct. The 864 bp tfdA gene in the construct is driven by the CaMV35S core promoter and linked to the nos gene terminator. Transformed embryogenic cells were selected on LTMM containing 350 mg l⁻¹ kanamycin and induced to develop somatic embryos on NN medium supplemented with 10 μM IASP, 8 μM NOA, 1 μM TDZ, 1 μM ABA and 2.5 g l⁻¹ activated charcoal (Embryo Development and Maturation Medium, EDMM). Somatic embryos were germinated and converted into plantlets in ½ MS (Murashige & Skoog, 1962) medium containing 0.5 μM BA+0.025 μM NAA. PCR analysis of regenerated plantlets with tfdA-specific primers showed they contained the tfdA gene. The expression of the tfdA gene in the transgenic plants was demonstrated by their 2,4-D resistance during spray tests. Transgenic plants survived treatment with 0.5, 5, and 10 kg ha⁻¹ of 2-ethylhexyl ester of 2,4-D (LV400, Growmark Inc., USA). These rates killed non-transgenic plants.

Spray tests utilized applications of 2,4D corresponding to field application rates of 0, 0.5, 5 and 10 kg/ha of a commercial herbicide preparation (LV400, Growmark Inc). After spraying, the transgenic plant and wild type plants were allowed to dry and then transferred to an isolation greenhouse, and they were observed for damage over a period of three weeks. The wild type plants showed signs of damage within two hours of spraying and they were all dead within one week. While the transgenic plant showed minor, short lived injury (leaf epinasty) for up to seven days at the 2 higher doses of 24D, there was full recovery, with normal growth at the end of two weeks.

The original vine of ‘Improved Chancellor’ arose from selection among embryogenic cells developed in vitro. The cells had been transformed with the tfdA gene. About 20 plants were regenerated between 2002 and 2003; ‘Improved Chancellor’ was selected from these. It was then micropropagated by cuttings. Those resulting plants were stable and typical of the original vine. ‘Improved Chancellor's resistance to 2,4-D was confirmed in 2004 in the original plant and in asexually propagated material from the original plant. Subsequent asexual propagations of the variety have also proven stable with true to type plants.

### Comparison with Parental Cultivar

The new grape plant named ‘Improved Chancellor’ resembles the parent grape, from which it was produced by genetic modification, but it differs in that it is significantly less sensitive to herbicide 2,4-D. The fruit color, flavor and texture is expected to be similar to the original grape. Vigor of the new variety is expected to be the same as the parent plant in absence of herbicide. However, vigor of ‘Improved Chancellor’ may increase in the presence of herbicide.

### DESCRIPTION OF THE FIGURES

FIG. 1 Shows canes, leaves, and tendrils of ‘Improved Chancellor’ (left) in comparison to the parent (right)

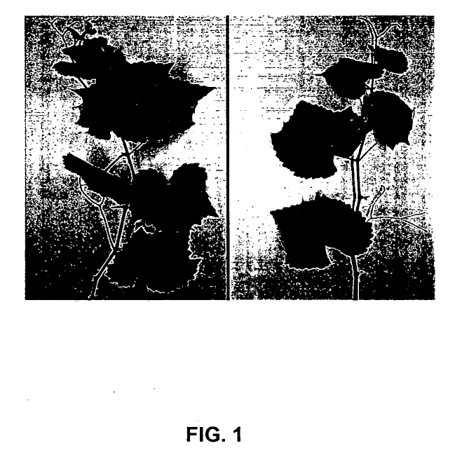

FIG. 2 Shows fruit clusters of parental ‘Chancellor’ at harvest.

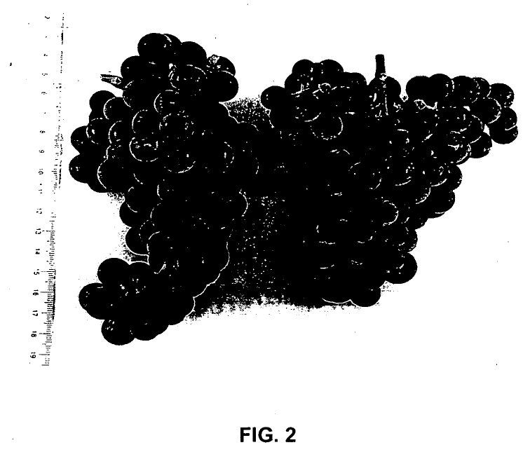

### DETAILED BOTANICAL DESCRIPTION OF THE INVENTION

The following description of grapevine contains references to color names taken from the Ridgeway Color Standards and Color Nomenclature (1912, Hoen and Co., Baltimore, Md.). Descriptors used herein conform to those set forth by the International Board for Plant Genetic Resources Institute Grape Descriptors (Vitis spp.) of 1983 and/or 1997 which were developed in collaboration with the Office International de la Vigne et du Vin (OIV) and the International Union for the Protection of New Varieties of Plants (UPOV) and published in Descriptors for Grapevine (Vitis spp.) (Anonymous, International Plant Genetic Resources Institute, 1997, ISBN 92-9043-352-3).

Descriptions of the new invention apply to vines of ‘Improved Chancellor’ grown in an isolation greenhouse at the University of Illinois at Urbana-Champaign in the year 2005. These vines were in their first year of growth having been transplanted from in vitro to the greenhouse in December 2004. The parent clones (control) were growing on their own roots at the University of Illinois pomology farm. The descriptions of the parent plants are believed to apply generally to the new variety grown under similar circumstances elsewhere:
Vine:
General:
   Vigor.—Too young to give valid data.
   Productivity.—unavailable.
   Hardiness.—unavailable.
   Rootstock.—none.
Leaves:
Mature leaves:
   Average blade length.—8.6 cm.
   Average blade width.—12.5 cm.
   Size of blade.—large.
   Shape.—pentagonal.
   Anthocyanin coloration of main veins on the upper side of the blade.—Present at base of veins on mature leaves.
   Mature leaf profile.—undulating.
   Blistering surface of blade upper surface.—absent.
   Leaf blade tip.—curved downward.
   Margins.—serrate.
   Apex.—acuminate.
   Base.—sagittate.
   Thickness.—medium.
   Undulation of blade between main and lateral veins.—medium.
   Shape of teeth.—conical, both sides convex.
   Length of teeth.—3.8 mm.
   Ratio length/width of teeth.—about 1:1.6.
   General shape of petiole sinus.—Y-shaped.
   Tooth at petiole sinus.—absent.
   Petiole sinus limited by veins.—absent.
   Shape of upper lateral sinus.—open Y-shaped.
   Prostrate hairs between veins on lower surface of blade.—absent.
   Erect hairs between veins on lower surface of blade.—absent.
   Prostrate hairs on main veins on lower surface of blade.—present.
   Density of erect hairs on main veins on lower surface of blade.—sparse.
   Prostrate hairs on main veins on upper surface of blade.—absent.
Upper surface:
   Summer color.—Civette green, 31′ Y-G. Plate XVIII.
   Autumn color.—data to be collected in fall.
   Surface texture.—smooth.
   Surface appearance.—medium glossy.
   Goffering of blade.—medium on mature leaves.
Lower surface:
   Summer color.—Mineral green, 31″. Y-G. i Plate XVIII.
   Autumn color.—data to be collected in fall.
   Anthocyanin coloration of main veins on lower leaf surface.—Light grayish vinaceous, 9′″ OR-O Plate XXXIX.
   Glossiness.—low.
   Pubescence.—mildly present.
   Surface texture.—medium leathery.
   Surface appearance.—dull.
Petiole:
   Length of petiole.—6.4 cm.
   Diameter.—2.3 mm.
   Color.—most of petiole — Bright Clalcedony yellow, 29′ YG-Y i Plate XVII but with streaks of Jasper red, 3′. O-R. i Plate XIII in leaves exposed to the sun.
   Length of petiole compared to middle vein.—about the same.
   Density of prostrate hairs on petiole.—sparse on young leaves; absent on mature leaves.
   Density of erect hairs on petiole.—dense on young leaves.
   Shape of base of petiole sinus.—mostly open, with inside outline ovate.
Tendrils:
   Number.—tendrils at all nodes above node #2; abort on older growth.
   Length.—14.8 cm.
   Diameter.—1.4 mm.
   Texture.—smooth.
   Color.—Mineral green with occasional Brown vinaceous, 5′″. OO-R Plate XXXIX.
Trunk:
   Trunk circumference.—0.4 cm. at 1 meter height. Mean of 10 plants.
   Shape.—medium.
   Surface texture.—sooth — canes still young.
   Outer bark color.—Vinaceous Tawny, 11″ orange Plate XXVII.
Canes:
   Shape.—broadly elliptical.
   Internode length.—6.7 cm. Mean of 10 canes counting nodes # 3-6.
   Width at node.—About 0.75 cm. Mean of 10 canes measuring nodes # 3-6.
   Cross section.—circular.
   Surface.—smooth.
   Main color.—Bright Clalcedony Yellow, 25′ YG-Y-i Plate XVII.
   Lenticels.—inconspicuous.
   Erect hairs on nodes.—absent.
   Erect hairs on internodes.—absent.
   Growth of axillary shoots.—moderately prolific.
   Shape.—circular to broadly elliptical.
   Number.—lateral shoots generally develop at all nodes above node # 5.
   Length.—grow to about 1 m in the field.
   Diameter.—3-5 mm in the field.
   Internode length.—5-7 cm in the field.
   Color.—Mineral green with occasional red coloration on surfaces facing the sun in the field.
Buds:
   Shape.—conical.
   Length.—4 mm. — mean of 10 buds node #10 from 10 shoots.
   Width.—3.2 mm. mean of 10 buds node #10 from 10 shoots.
   Color.—Vinaceous Tawny, 11″ orange Plate XXVII.
   Basal buds.—fruitful, 1-2 clusters per shoot — on parent field ‘Chancellor’.
Flowers: Data from mature field parent ‘Chancellor’ plants
General:
   Flower sex.—perfect.
   Length of first inflorescence.—4.7 cm. — Mean of 7 inflorescences.
   Position of first flowering nodes.—#2-3.
   Number of inflorescences per shoot.—# 2-3.
   Pedicel length.—2.3 mm. — Mean of 10 pedicels.
   Calyptra color.—Oil green, 27-G-Y-k Plate V.
   Ovary length.—1.8 mm. — Mean of 10 ovaries.
   Ovary width.—1.4 mm. — Mean of 10 ovaries.
   Ovary color.—Light fluorite green, 33″. GY-G.d, Plate XXXII.
   Filament length.—2.1 mm. Composite mean of 2 filaments each from 10 flowers.
   Filament color.—Grayish white.
   Anther length.—1.3 mm. Composite mean of 2 anthers each from 10 flowers.
   Anther color.—Cream color 19′. YO-Y. f Plate XVI.
   Date of full bloom.—about April 30 in Urbana, Ill.
Fruit: (All fruit data for parent ‘Chancellor’ field-grown.)
General:
   Ripening period.—August 30-September 11 at Urbana, Ill. (Data from 3 years of parent ‘Chancellor’ harvested in the field).
   Date of ripening.—about August 30-September 11 in Urbana, Ill.
   Use.—wine.
   Keeping quality.—good.
   Insects.—typical of Vitis vinifera.
   Diseases.—typical of Vitis vinifera.
   Herbicide resistance.—significantly greater than parent ‘Chancellor” which was not genetically modified to contain and express the tfdA coding sequence, especially with respect to 2,4-D spray drift from surrounding agricultural land.
   Shipping quality.—good.
   Date of first harvest.—August 30 at Urbana, Ill. Data from 3 years of ‘Chancellor harvested in the field.
   Solids — sugar.—High, 20.3 brix at full maturity. (parent Chancellor grapes, Dr. Terry Bates, 2002. Cornell University, Fredonia Vineyard Laboratory).
   Refractometer test.—20.3 brix for parent Chancellor grape.
Cluster: Data from mature field grown parent ‘Chancellor’
   Bunch size.—medium.
   Bunch length (peduncle excluded).—About 15.3 cm. — Mean of 7 clusters.
   Bunch width.—5.6 cm. — Mean of 7 clusters Bunch weight (natural). — 127.3 g. — Mean of 7 clusters.
   Bunch density.—dense.
   Number of berries in a cluster.—102 — Means of 7 clusters.
   Form.—Long cylindrical.
Peduncle:
   Length of peduncle.—3-4 cm.
   Lignification of peduncle.—strong in upper 2 cm.
   Color.—Brown with some red.
Berry:
   Size.—medium.
   Uniformity of size.—uniform.
   Berry weight (natural).—1.6 g. — Mean of 10 berries Shape. — Roundish.
   Presence of seeds.—yes, avg. wt.=124.3 mg. — Mean of seeds from 10 berries each containing 3 seeds.
   Cross section.—circular.
   Dimensions.—longitudinal axis about 1.4 cm.; horizontal axis about 1.3 cm. — Means of 10 berries.
   Skin color (without bloom).—Dark purplish gray, 67′″″. V-R. m Plate LIII.
   Coloration of flesh.—translucent.
   Juiciness of flesh.—medium juicy.
   Berry firmness.—very firm.
   Particular flavor.—typical vinifera.
   Bloom (cuticular wax).—strong.
   Pedicel length.—10.1 mm. — Mean of 10 berries.
   Berry separation from pedicel.—moderate.
Skin: (all data from parent ‘Chancellor’ grown in the field)
   Thickness.—medium.
   Texture.—tender.
   Reticulation.—absent.
   Roughness.—smooth.
   Tenacity.—tenacious.
   Tendency to crack.—resistant.
Seeds:
   Number per berry.—2-3 seeds/berry.
   Seed weight.—57 mg/seed. — Mean of 25 seeds.
   Seed color.—Verona brown, 13′. OY-O. m Plate XXIX.

### REFERENCES

Anonymous. International Plant Genetic Resources Institute, 1997, ISBN 92-9043-352-3
Murashige, T. and F. Skoog. 1962. A revised medium for rapid growth and bio-assays with tobacco tissue cultures. Physiologia Plantarum, 15: 473-497.
Nistch, J. P. and C. Nistch. 1969. Haploid plants from pollen grains. Science, 163: 85-87.
Ridgeway, R. 1912. Color standards and color nomenclature. A. Hoen and Company, Baltimore, Md.
Skirvin, R. M., M. A. Norton, A. G. Otterbacher, R. Mulwa, B. Shoemaker, B. Aly, and E. Wahle. 2005. Grape varieties for the different regions of Illinois—2005. Proceedings 2005 Illinois Small Fruit & Strawberry Schools. NRES 7:29-40.

What is claimed is:

1. A new and distinct variety of grape plant named ‘Improved Chancellor’ as herein illustrated and described.
